(19) United States
(12) Patent Application Publication
(10) Pub. No.: US 2007/0225718 A1
(43) Pub. Date: Sep. 27, 2007

(54) Rotolock cervical plate locking mechanism

(76) Inventor: Michael D. Ensign, Salt Lake City, UT (US)

Correspondence Address: RADER, FISHMAN & GRAUER PLLC, 10653 SOUTH RIVER FRONT PARKWAY, SUITE 150, SOUTH JORDAN, UT, 84095, US

(21) Appl. No.: 11/726,869

(22) Filed: Mar. 21, 2007

Related U.S. Application Data
(60) Provisional application No. 60/784,627, filed on Mar. 22, 2006.

(51) Int. Cl. A61F 2/30

(52) U.S. Cl. 606/69

(57) ABSTRACT

According to one exemplary embodiment, an orthopedic bone fixation device for stabilizing a plurality of bone segments includes a bone plate and a screw assembly. The bone plate includes a body defining at least one thru-bore, wherein the thru-bore is defined to include a central cavity, the central cavity includes a split ring, a compliant member, or another positionable element configured to modify an exit diameter of the thru-bore. Additionally, an actuation member is coupled to the bone plate. According to one exemplary embodiment, actuation of the actuation member, either by rotation, sliding, or the like, causes the actuation member to engage the positionable member, thereby modifying the exit diameter of the thru-bore. Further, the screw assembly is configured to be coupled to the bone plate, wherein the screw assembly includes a bone screw having a head section and a thread section. When actuated, the positionable element is configured to reduce the exit diameter of the thru-bore sufficient to interfere with the head section of the bone screw, thereby preventing the screw from backing out.

### RELATED APPLICATIONS

This application claims the benefit under 35 U.S.C. §119(e) of U.S. Provisional Patent Application No. 60/784,627 filed Mar. 22, 2006 titled “Rotolock Cervical Plate Locking Mechanism,” which provisional application is incorporated herein by reference in its entirety.

### FIELD

The present system and method relate to bone fixation devices. More particularly, the present system and method provide for an orthopedic system including a plate, a screw system, and a complete system including the plate system, the screw system, and the screw retention system.

### BACKGROUND

In the treatment of various spinal conditions, including the treatment of fractures, tumors and degenerative conditions, it is necessary to secure and stabilize the anterior column of the spine following removal of a vertebral body or part. Various devices for internal fixation of bone segments in the human or animal body are known in the art.

Following such removal made using a thoracotomy, thoracoabdominal or retroperitoneal approach, the normal anatomy is reconstructed using tricortical iliac crest or fibular strut grafts. Not only are removals performed on the thoracic spine, as is the case for the above procedures, but also the cervical spine. Once bone matter is removed, it is then necessary to secure and stabilize the graft, desirably in such a manner as to permit rapid mobilization of the patient. Such objectives can be accomplished by a bone plate. However, to accomplish this service in the optimum manner, it is necessary that the plate be reasonably congruent with the bone to which it is applied, that it have as low a profile as possible, that it be firmly secured to the spinal column so that it is not torn out when the patient places weight and stress upon it and that it be capable of placement and fixation in a manner that is convenient for the surgeon.

In this context it is necessary to secure the plate to the spinal body and also, in some cases, to the graft. Conventionally, such attachment would be by the use of screws driven through screw holes in the plate into the bone. However, when stabilizing the position of cervical vertebrae, the plate is designed to lie near and posterior to the esophagus of the patient. Due to its relative location to the esophagus and other connective tissue, if the screw securing the plate to the cervical spine backs out, the screw could irritate or even pierce the esophagus, resulting in pain, infection, and/or possible death of the patient. Consequently, anti-back out mechanisms are desired in the orthopedic plate industry.

### SUMMARY

According to one exemplary embodiment, an orthopedic bone fixation device for stabilizing a plurality of bone segments includes a bone plate and a screw assembly. The bone plate includes a body defining at least one thru-bore, wherein the thru-bore is defined to include a central cavity, the central cavity includes a split ring, a compliant member, or another positionable element configured to modify an exit diameter of the thru-bore. Additionally, an actuation member is coupled to the bone plate. According to one exemplary embodiment, actuation of the actuation member, either by rotation, sliding, or the like, causes the actuation member to engage the positionable member, thereby modifying the exit diameter of the thru-bore. Further, the screw assembly is configured to be coupled to the bone plate, wherein the screw assembly includes a bone screw having a head section and a thread section. When actuated, the positionable element is configured to reduce the exit diameter of the thru-bore sufficient to interfere with the head section of the bone screw, thereby preventing the screw from backing out.

### BRIEF DESCRIPTION OF THE DRAWINGS

The accompanying drawings illustrate various exemplary embodiments of the present system and method and are a part of the specification. Together with the following description, the drawings demonstrate and explain the principles of the present system and method. The illustrated embodiments are examples of the present system and method and do not limit the scope thereof.

FIG. 1 is a side view of an assembled cervical plate system, according to one exemplary embodiment.

FIG. 2 is an exploded view illustrating the components of the screw assembly and bone plate of the exemplary embodiment illustrated in FIG. 1.

FIG. 3 is a top view of a traditional bone plate, according to various exemplary embodiments.

FIG. 4A through 4F are various views of a rotationally locked cervical plate system and its individual components, according to one exemplary embodiment.

FIGS. 5A through 5D are various views of a rotationally locked cervical plate system and its individual components, according to an alternative exemplary embodiment

FIG. 6 is a flow chart illustrating a method of securing an orthopedic plate, according to one exemplary embodiment.

FIGS. 7A and 7B are a top and a cross-sectional view of a rotationally locked cervical plate design in an un-locked position, according to one exemplary embodiment.

FIGS. 7C and 7D are a top and a cross-sectional view of a rotationally locked cervical plate design in a locked position, according to one exemplary embodiment.

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings. Throughout the drawings, identical reference numbers designate similar but not necessarily identical elements.

### DETAILED DESCRIPTION

The present specification describes a system and a method for coupling an orthopedic plate to one or more bones while preventing back-out of the fastener. Further, according to one exemplary embodiment, the present specification describes the structure of an orthopedic plate system that selectively constricts the diameter of a thru-bore in the orthopedic plate, thereby preventing back-out of a screw while positionally fixing bone segments. Further details of the present exemplary system and method will be provided below.

By way of example, orthopedic plate systems may be used in the treatment of various spinal conditions. As mentioned, when applied to stabilize the position of cervical vertebrae, the plate portion of the orthopedic plate system is designed to lie near and posterior to the esophagus of the patient. Due to its relative location to the esophagus and other connective tissue, the top surface of the plate portion may be smooth and free of sharp corners to prevent irritation or piercing of the esophagus and surrounding tissue. Further, in order to prevent irritation and/or piercing, any connection hardware that is used to couple the plate portion to the cervical vertebrae should remain below or even with the top surface of the plate portion.

If the screw or other fastener securing the plate portion to the cervical spine backs out or otherwise protrudes above the top surface of the plate portion, the screw could irritate or even pierce the esophagus, resulting in pain, infection, and/or possible death of the patient. Consequently, the present exemplary system and method provide an orthopedic plate system including a bone plate with thru-bores. According to the exemplary embodiments disclosed below, the exit diameter of the thru-bores may be selectively modified to secure one or more bone screws with in the thru-bores, thereby preventing the bone screws from backing out.

Moreover, the present exemplary system and method provides anti-back out protection via an integral or immediately coupled component of the bone plate. Consequently, head height of the bone screw may remain unchanged.

In the following description, certain specific details are set forth in order to provide a thorough understanding of various embodiments of the present orthopedic plate system and method. However, one skilled in the relevant art will recognize that the present exemplary system and method may be practiced without one or more of these specific details, or with other methods, components, materials, etc. In other instances, well-known structures associated with orthopedic plate systems have not been shown or described in detail to avoid unnecessarily obscuring descriptions of the present exemplary embodiments.

Unless the context dictates otherwise, throughout the specification and claims which follow, the word “comprise” and variations thereof, such as, “comprises” and “comprising” are to be construed in an open, inclusive sense, that is as “including, but not limited to.”

The term “compliant mechanisms” relates to a family of devices in which integrally formed flexural members provide motion through deflection. Such flexural members may therefore be used to replace conventional multi-part elements such as pin joints. Compliant mechanisms provide several benefits, including backlash-free, wear-free, and friction-free operation. Moreover, compliant mechanisms significantly reduce manufacturing time and cost. Compliant mechanisms can replace many conventional devices to improve functional characteristics and decrease manufacturing costs. Assembly may, in some cases, be obviated entirely because compliant structures often consist of a single piece of material.

Reference in the specification to “one embodiment” or “an embodiment” means that a particular feature, structure, or characteristic described in connection with the embodiment is included in at least one embodiment. The appearance of the phrase “in one embodiment” in various places in the specification are not necessarily all referring to the same embodiment. Furthermore, the particular features, structures, or characteristics may be combined in any suitable manner in one or more embodiments.

### Exemplary Structure

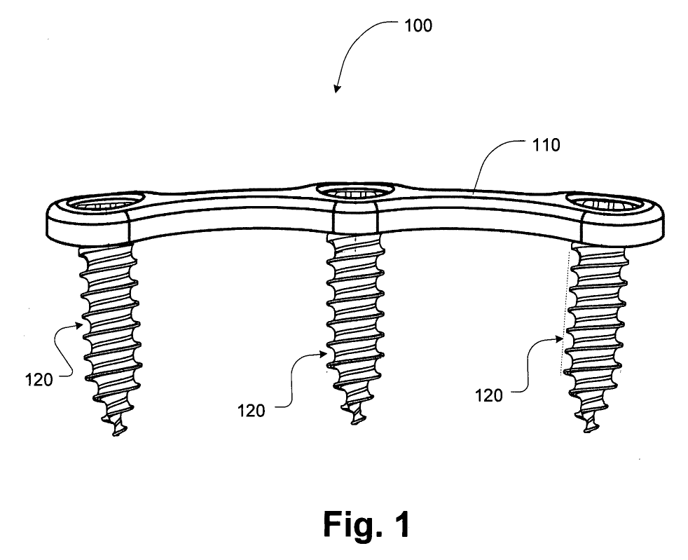

FIG. 1 illustrates a traditional assembled cervical plate system (100), according to one exemplary embodiment. As illustrated, the traditional cervical plate system (100) includes a number of components including, but in no way limited to, a bone plate (110) and at least one screw (120) coupled to the bone plate (110). According to the exemplary embodiment illustrated in FIG. 1, the screws (120) are configured to be securely coupled to a patient's bone(s) while securely coupling to the bone plate (110) to provide structural and positional stability while preventing issues with the screw assembly backing out. Further, as illustrated in FIG. 1, the exemplary cervical plate system (100), when assembled, maintains the highest point of the screw (120) below the highest surface of the bone plate (110).

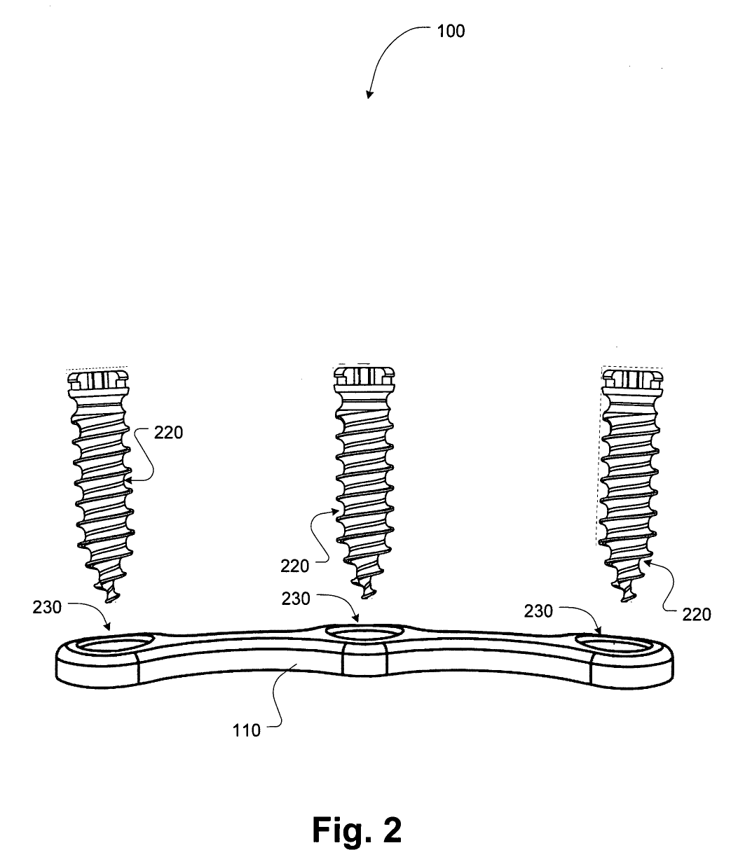

FIG. 2 is an exploded view of the traditional cervical plate system (100). The screw assembly (120) is selectively inserted into the thru bore(s) (230) formed in the exemplary bone plate (110). As mentioned, when fully engaged, the traditional cervical plate system (100) is able to maintain a relatively low profile while providing structural support. However, there is little or no structure for preventing screw back out. That is, the thru bore(s) merely receive and house the head portion of the screw assembly (120) and provide little or no resistance to the screw backing out.

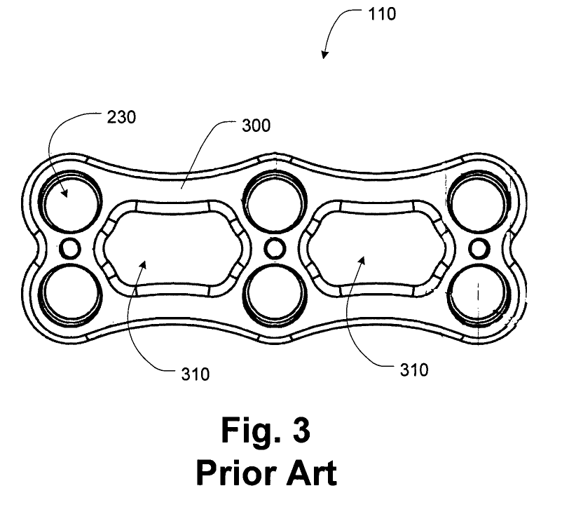

FIG. 3 illustrates a traditional bone plate (110), according to one exemplary embodiment. As shown, the bone plate generally includes a main plate body (300) having a number of material cut-out(s) (310) and thru-bore(s) (230) formed therein. The plate body (300) of the bone plate (110) may be slightly curved to follow the shape of a spinal column and may be formed out of any number of biocompatible metals including, but in no way limited to, stainless steel, titanium, or a titanium alloy. Moreover, the construction of the plate body (300) may be made of non-metal materials including, but in no way limited to, carbon reinforced Polyetheretherketone (PEEK), and the like. Additionally, as illustrated in FIGS. 3A and 3B, the plate body (300) has a beveled rounded periphery to eliminate any sharp or abrupt edges that could potentially be damaging to surrounding tissue.

The material cut-out(s) (310) formed in the plate body (300) may serve a number of purposes. According to one exemplary embodiment, the material cut-out(s) (310) may be designed to eliminate superfluous material, thereby reducing the overall weight of the bone plate (110), while maintaining the desired structural integrity. Additionally, the various material cut-out(s) (310) may be configured to facilitate handling of the bone plate (110) during installation or removal with a tool such as, but in no way limited to, forceps. Further, the material cut-out(s) (310) may also provide functional access to tissue and/or bone located behind an installed bone plate (110) without necessitating removal of the plate.

However, as illustrated in FIG. 3, the traditional bone plate (110) is void of any significant back out prevention feature, independent of the screw structure. Consequently, specialized screw assemblies such as those disclosed herein can be used in conjunction with the traditional bone plate (110) to prevent screw back out.

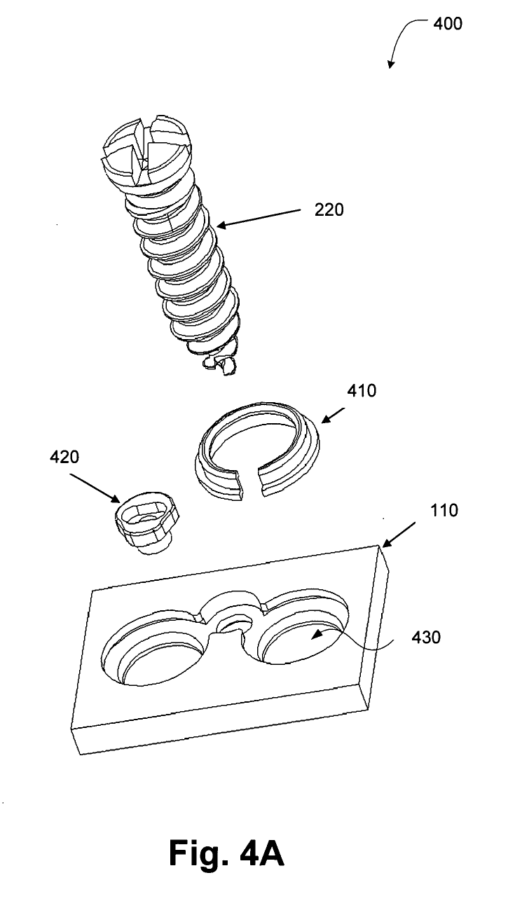

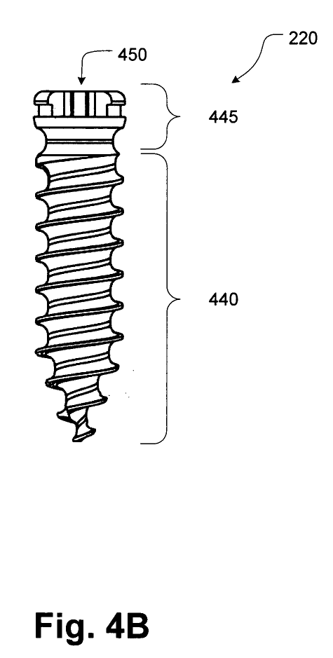

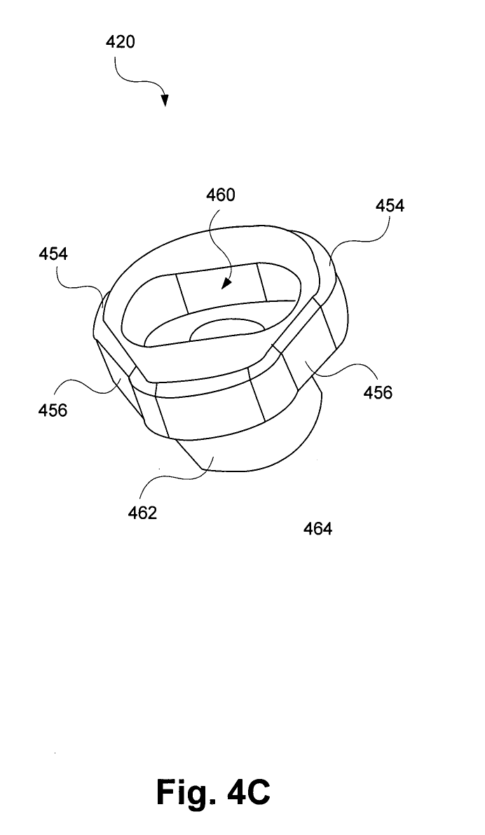

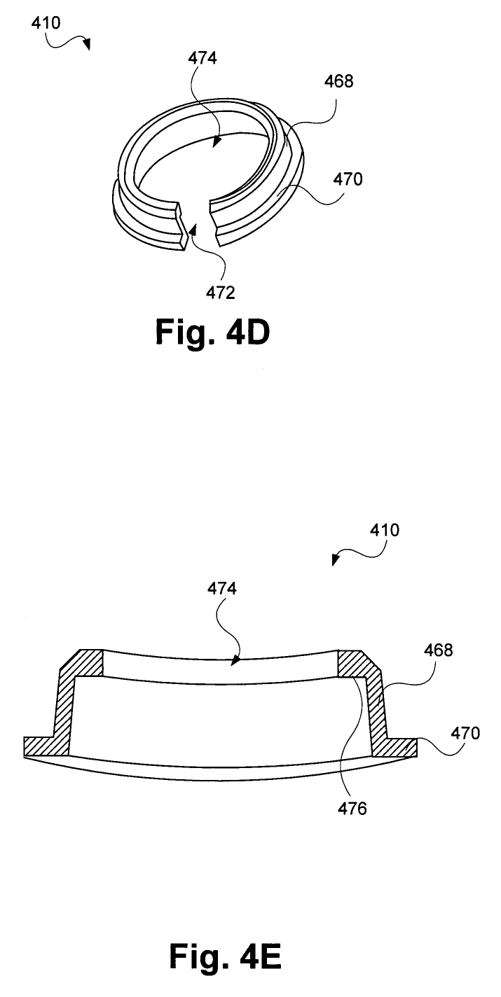

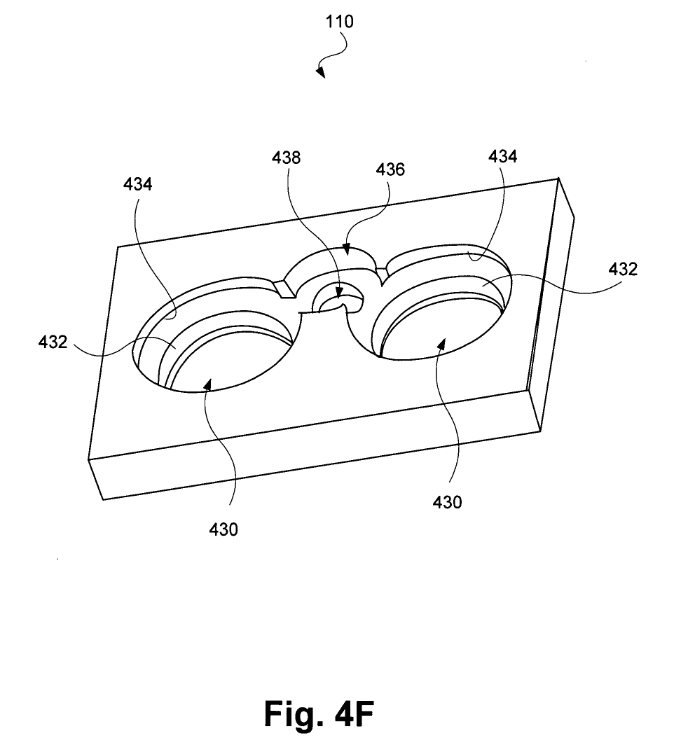

In contrast to the traditional cervical bone plate, FIG. 4 illustrates a portion of a rotationally locking cervical plate system (400), according to one exemplary embodiment. As illustrated in FIG. 4, the present exemplary rotationally locking cervical plate system (400) includes a cervical plate (110) including a number of thru-bores (430) for receiving an orthopedic fastener. Additionally, as shown, the present exemplary system (400) includes a cammed actuator (420) configured to be disposed in the cervical plate (110) adjacent to the thru bore (430). Moreover, the present system includes a compressible retention member (410) configured to selectively vary the exit diameter of the thru-bore (430) based on an actuation position of the cammed actuator (420). Furthermore, as is present in traditional cervical plate systems, an orthopedic fastener (220) is included to secure the present plate (110) to a desired site. Further details of the present exemplary rotationally locking cervical plate system (400) will be provided below with reference to FIGS. 4B through 4E.

An exemplary orthopedic fastener (220) that may be used with the present rotationally locking cervical plate system (400) is illustrated in FIG. 4B. While a traditional bone screw (220) is illustrated in FIG. 4B, and is used for simple explanation herein, the present rotationally locking cervical plate system (400) may be used with any number of orthopedic fasteners having a protruding member, such as a head. Particularly, as illustrated in FIG. 4B, an exemplary bone screw is shown. As shown, the exemplary bone screw (220) includes a head portion (445) and a threaded portion (440). As is known in the art, the threaded portion (440) may include, but is in no way limited to a self-tapping thread configured to remove bone and other material as it is driven into a desired site. Additionally, according to one exemplary embodiment, the head portion (445) of the bone screw or other orthopedic fastener may assume any number of shapes including, but in no way limited to, a circular shape, an oval shape, a quadrilateral based shape, or the like. Additionally, as illustrated in FIG. 4B, the orthopedic fastener (220) may include any number of driving features (450) configured to aid in driving the orthopedic fastener into a desired site. For example, according to one exemplary embodiment, the driving feature of the orthopedic fastener (220) may include, but is in no way limited to, a hex-head, a Phillips head, a flat head, and the like.

FIG. 4C illustrates the features of the present exemplary cammed actuator (420), according to one exemplary embodiment. As shown, the cammed actuator (420) includes a main body having a number of lobes. Particularly, as illustrated in FIG. 4C, the cammed actuator is a heart-shaped actuator including a plurality of raised engagement lobes (454) on an upper half of the cammed actuator (420), and a plurality of recessed converging surfaces or lobes (456) on a lower half of the actuator (420). Additionally, according to the illustrated exemplary embodiment, an actuating orifice (460), such as a slit or other profile configured to receive an instrument, is defined in the upper surface of the actuator (420). Furthermore, a pivot element (462) is projecting from the bottom of the cammed actuator (420) and can include a coupling element (464) for coupling the actuator to the bone plate (110). According to one exemplary embodiment, the coupling element (464) is configured to receive a backing element (not shown) or some other positioning element configured to maintain the cammed actuator rotatably coupled to the bone plate (110). The presently illustrated cammed actuator allows for a single actuator to lock or disengage two orthopedic fasteners simultaneously. While the present exemplary embodiment is illustrated as including a cammed actuator (420) configured to simultaneously couple two orthopedic fasteners, any number of lobes and recesses may be formed to couple various numbers of fasteners.

As illustrated in the exemplary embodiment of FIG. 4A, the cammed actuator (420) is rotatable coupled to the bone plate (110) interposed between a plurality of thru-bores (430). According to one exemplary embodiment, the cammed actuator (420) is configured to have two main positions: locked and unlocked. When the cammed actuator is in the unlocked position, according to one exemplary embodiment, the two raised engagement lobes (454) are not forced upon the periphery of the compressible retention members (410). However, when in the locked position, the cammed actuator (420) is rotated approximately 180 degrees, according to one exemplary embodiment, and the two raised engagement lobes (454) are actuated against the compressible retention members (410).

FIGS. 4D and 4E illustrate a perspective view and a cross-sectional view of a compressible retention member (410), according to one exemplary embodiment. As illustrated, the retention member (410) may be a split-ring (410). As illustrated, the split ring (410) includes a main ring body (468) having a generally circular profile. Additionally, as shown, the split ring (410) can include an externally protruding annular retention flange. As will be described below, the annular retention flange (470) may correspond to any number of features present in a thru-bore (430) of the bone plate (110) to be used to maintain the split ring (410) within the bone plate (110). Additionally, according to one exemplary embodiment, the ring body (468) includes a back-out projection (476) that projects inward towards the center of the split ring (410) and may be selectively imposed into the pathway of an orthopedic fastener (220) to prevent the fastener (220) from backing out from the plate (110). Furthermore, the split ring (410) includes a split (472) allowing for the selective compression of the split ring by the cammed actuator (420; FIG. 4C), as will be described in detail below. According to one exemplary embodiment, the split ring (410) also defines a thru-bore (474) that, according to one exemplary embodiment, has a diameter greater than the diameter of the head portion (445) of the orthopedic fastener (220) when the split ring (410) is in its relaxed state. However, when acted upon by a cammed actuator (420; FIG. 4C), the diameter of the split ring (410) disposed in the opening of the plate thru-bore (430) is less than the diameter of the head portion (445) of the orthopedic fastener (220).

FIG. 4E illustrates the present exemplary bone plate (110), according to one exemplary embodiment. As illustrated, the bone plate (110) includes a number of thru-bores (430) joined by a cam recess (436). According to one exemplary embodiment, the cam recess (436) includes a cam retention port (438) configured to receive the pivot element (462; FIG. 4C) of the cammed actuator (420; FIG. 4C). Additionally, the internal wall of the thru-bores (430) contain a number of features. Particularly, the thru-bore (430) can include a ring retention undercut (434) configured to receive and mate with the annular retention flange (470; FIG. 4D) of the split ring (410; FIG. 4D), thereby coupling the split ring to the thru-bore. According to one exemplary embodiment, the split-ring (410) may be initially compressed and then inserted into the ring retention undercut (434). Furthermore, the bottom or exit diameter of the thru-bore (430) is less than the entrance diameter, thereby allowing for the passage of the bone screw (220) through the entrance diameter, but seating the head of the bone screw (220) on a screw seat (432). According to one exemplary embodiment, when the present exemplary system is assembled, a bone screw (110) may be introduced into the thru-bore (430) of the screw plate (110), past the relaxed compressible member (410) and into the screw head seat (432). The cammed actuator (420) may then be actuated to compress the compressible member (410) and introduce the back-out projection over the inserted screw head, thereby preventing the screw from backing out.

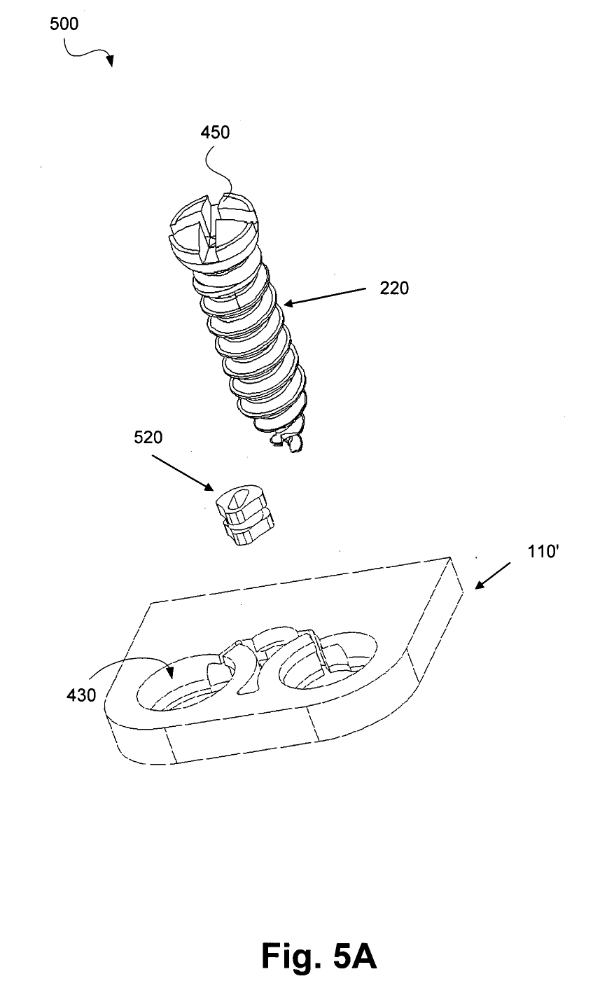

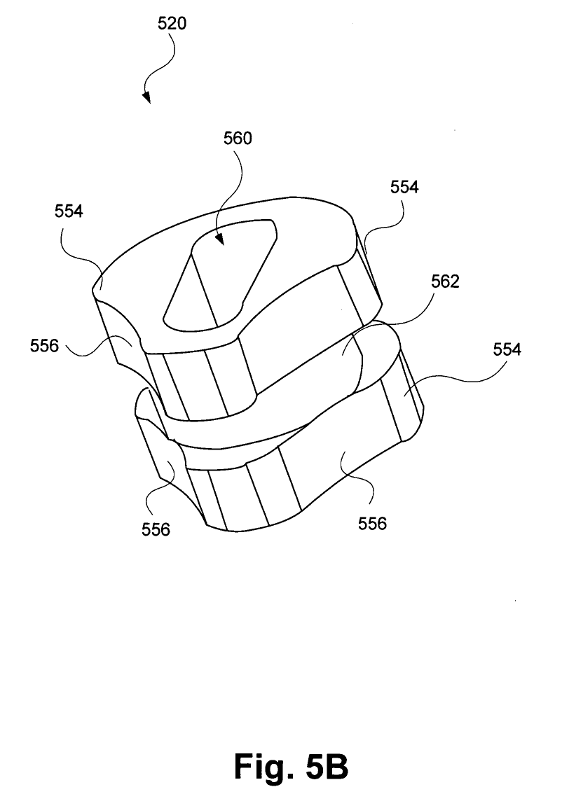

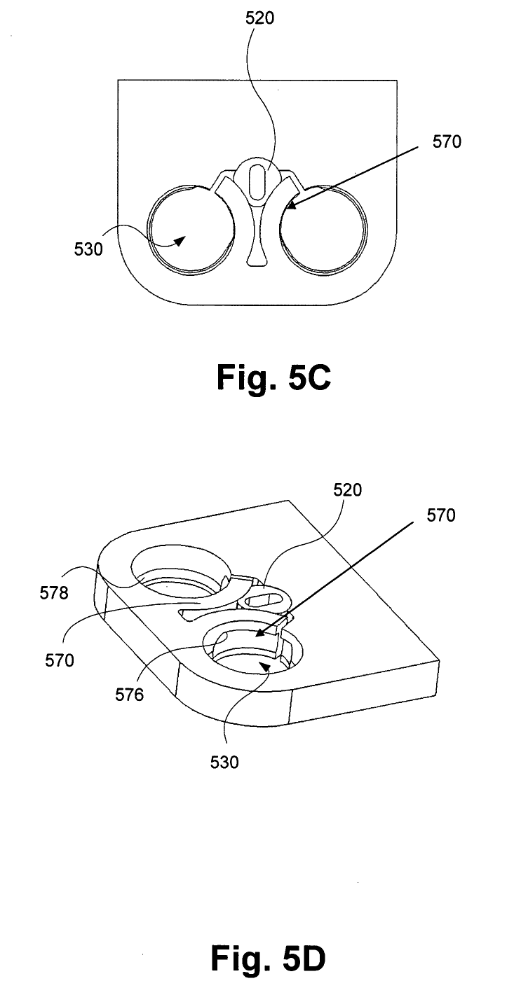

FIGS. 5A-5D illustrate an alternative configuration of the rotationally locking cervical plate system using another type of compressible member (410). As illustrated in FIG. 5A, an exemplary system can include a plate (110′) having a plurality of thru bores (430), as mentioned above. Additionally, the alternative configuration may use any number of orthopedic fasteners (220), as mentioned previously. However, in contrast to the previously illustrated rotationally locking cervical plate system, the present exemplary system (110′) includes a larger dual-cammed actuator (520) and a cantilevered compressible back-out member (570).

As shown in FIG. 5B, the dual-cammed actuator (520) includes substantially the same components as the previously mentioned cammed actuator (420). That is, the dual-cammed actuator (520) is a heart-shaped actuator including a plurality of raised engagement lobes (554) on an upper half of the cammed actuator (520), and a plurality of recessed converging surfaces or lobes (556) on a lower half of the actuator (520). Additionally, according to the illustrated exemplary embodiment, an actuating orifice (560), such as a slit or other profile configured to receive an instrument, is defined in the upper surface of the actuator (520). Furthermore, a pivot element (562) is disposed between the cammed surfaces.

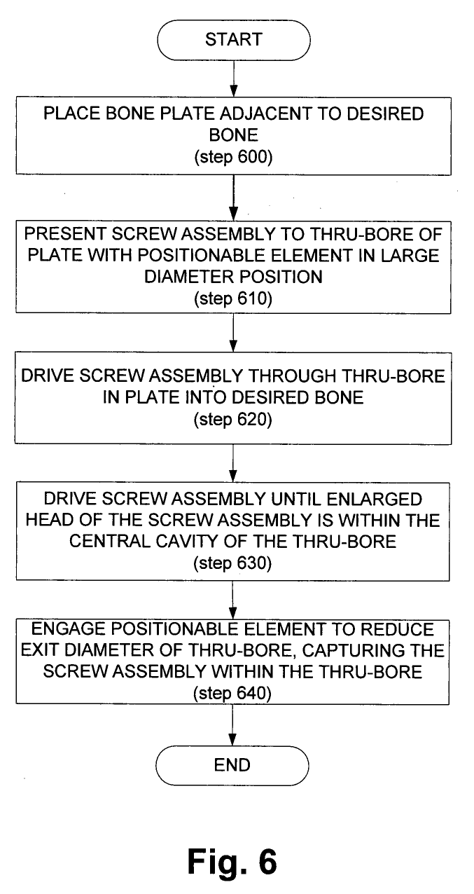

However, in contrast to the exemplary system illustrated in FIG. 4A, the present exemplary rotationally locking cervical plate system (500) includes cantilevered back-out members (570) in place of the previously used compressible split ring (410). According to the exemplary embodiment illustrated in FIGS. 5C and 5D, the cantilevered back-out members (570) are compliant and compressible members configured to perform the same operation as the split ring (410). That is, the cantilevered back-out members (570) include a back-out projection (576) extending towards the thru-bore (530). When a desired orthopedic fastener (220) is received, the cantilevered back-out member may receive a force transferred from the actuator (520) such that the back-out projection (576) interferes with the head portion of the orthopedic fastener (220) when disposed in the screw seat (578). An exemplary method of operation of the rotational lock cervical plate will be provided below with reference to FIGS. 6 through 8D.

### Exemplary Method

FIG. 6 illustrates a method for installing the exemplary cervical plate system including a rotational locking mechanism, according to one exemplary embodiment. As illustrated in FIG. 6, the present exemplary method for installing the cervical plate system includes placing the bone plate adjacent to one or more desired vertebral bones (step 7600). Once the bone plate is appropriately positioned, the screw assembly may then be presented to a thru-bore of the bone plate with the positionable element in a large diameter position (step 610). The screw assembly is then driven through the thru-bore in the bone plate into the desired vertebral bone (step 620) until the enlarged head of the screw assembly is within the central cavity of the thru-bore (step 630). Once the screw assembly is correctly positioned, the cammed actuator may be engaged to compress the compressible element and reduce the exit diameter of the thru bore, thereby capturing the screw assembly within the thru-bore (step 740). Further details of each step of the present exemplary method will be provided below with reference to FIGS. 8A through 8D.

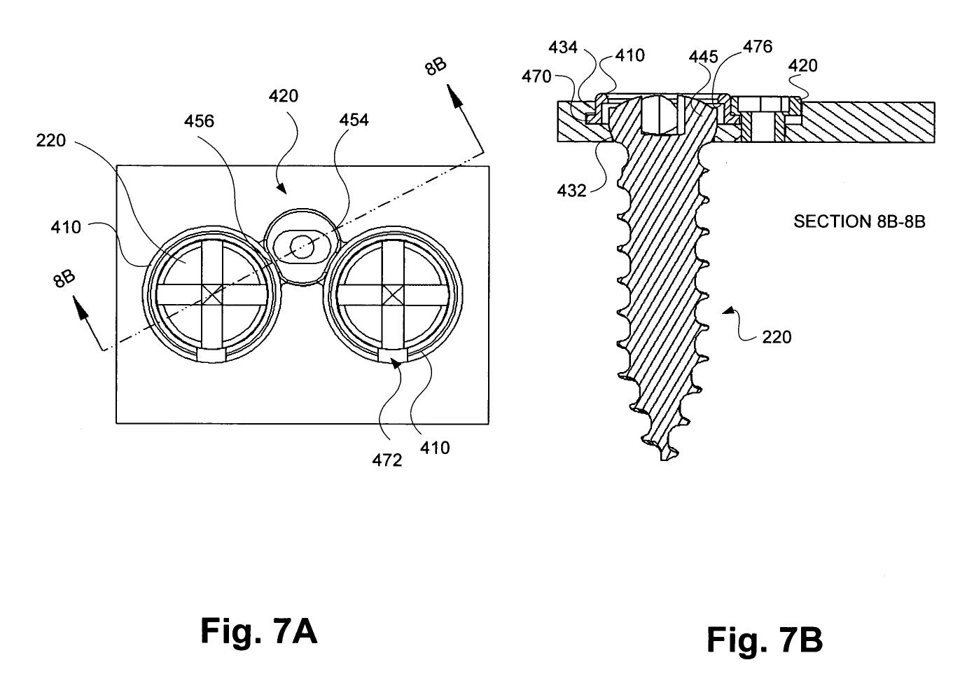

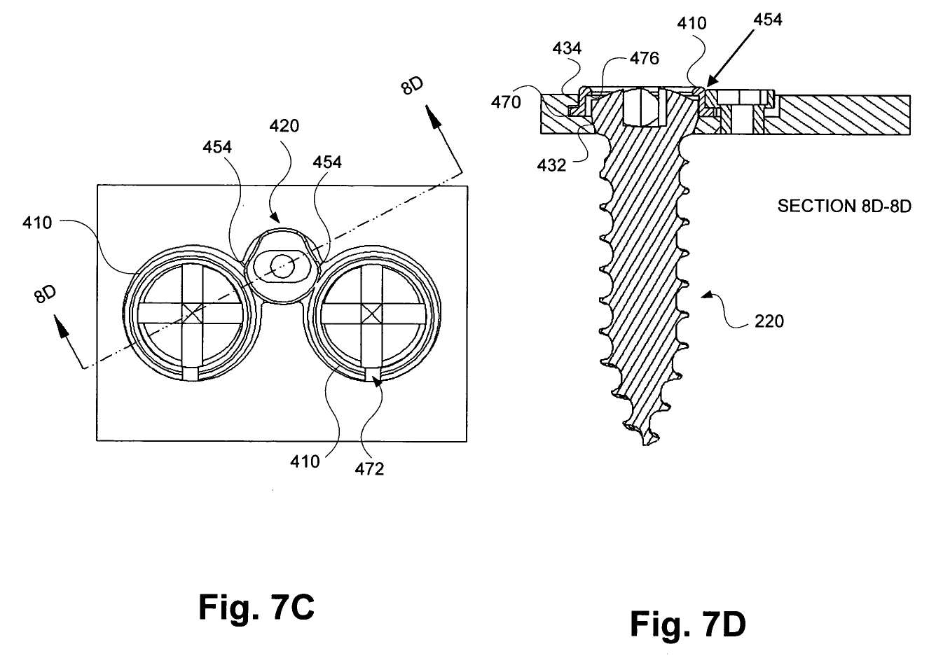

As illustrated in FIG. 7, the first step of the exemplary method is to place the plate adjacent to a desired vertebral bone (step 700). The placement of the bone plate relative to a vertebral bone in a patient may be pre-operatively determined based on a pre-operative examination of the patient's spinal system using non-invasive imaging techniques known in the art, such as x-ray imaging, magnetic resonance imaging (MRI), and/or fluoroscopy imaging, for example. Any additional preparation or work may be done on and around the desired vertebral bone prior to positionally orienting the bone plate.

With the bone plate appropriately positioned relative to a desired vertebral bone (step 700), the screw assembly may be presented to a thru-bore of the bone plate with the positionable element in a large diameter position (step 710). As shown in FIGS. 8A and 8B, the screw assembly may be delivered to the bone plate with the lock knob un-actuated, causing the positionable element to maximize the entry diameter of the thru-bore. Consequently, the screw assembly may be entered into the thru-bore without obstruction.

When presented, the screw assembly may then be driven through the thru-bore in the bone plate into a desired vertebral bone (step 720), as illustrated in FIGS. 8A and 8B. As illustrated in FIGS. 8A an 8B, the desired orthopedic fasteners (220) are driven through the thru-bore (430) while the cammed actuator (420) is in an unlocked position. In other words, the engagement lobes (454) are rotated away from the compressible retention member(s) (410) and the recessed surfaces (456) are in contact with the compressible retention members. Consequently, there is no interference for the head portion (445) of the orthopedic fastener (220) to pass there through. FIG. 8B also illustrates the annular retention flange (470) being maintained by the ring retention undercut (434).

Once the screw assembly is correctly positioned in the thru-bore (430), the compressible member (410) is engaged by the cammed activator (420) to reduce the exit diameter of the thru-bore (430), thereby capturing the orthopedic fastener within the thru-bore (step 740), as illustrated in FIGS. 8C and 8D, the back-out projection (476) impedes the removal of the orthopedic fastener (220). According to one exemplary embodiment, the cammed activator (420) is rotated approximately 180 degrees, causing the back out projection (476, 576) of the compressible member to be forced in over the orthopedic fastener. According to one exemplary embodiment, there can be a swept cutout in the cantilevered back-out member (570) or the compressible member (410) which mates with the head portion (445) of the orthopedic fastener (220).

While the present exemplary rotationally locking cervical plate system has been described, for ease of explanation only, in the context of a cervical plate system, the present exemplary systems and methods may be applied to any number of orthopedic fixtures. Specifically, the present screw back out prevention components may be used to couple any number of orthopedic apparatuses to a desired bone, for any number of purposes, as long as the connecting orthopedic apparatus includes a thru-bore substantially conforming with the configurations described herein.

In conclusion, the present exemplary systems and methods provide for coupling an orthopedic plate to one or more bones while preventing back-out of the fastener.

The preceding description has been presented only to illustrate and describe the present method and system. It is not intended to be exhaustive or to limit the present system and method to any precise form disclosed. Many modifications and variations are possible in light of the above teaching.

The foregoing embodiments were chosen and described in order to illustrate principles of the system and method as well as some practical applications. The preceding description enables others skilled in the art to utilize the method and system in various embodiments and with various modifications as are suited to the particular use contemplated. It is intended that the scope of the present exemplary system and method be defined by the following claims.

What is claimed is:

1. An orthopedic device comprising:
an implant member including a thru-bore;
a positionable element selectively defining an entry diameter of said thru-bore; and
an actuator coupled to said implant member;
wherein said actuator is configured to selectively impart a force on said positionable element to vary said entry diameter.

2. The orthopedic device of claim 1, wherein said positionable element comprises a split ring.

3. The orthopedic device of claim 1, wherein said positionable element comprises a compliant arm coupled to said implant member.

4. The orthopedic device of claim 1, wherein said actuator comprises a rotatable cam.

5. The orthopedic device of claim 4, wherein said rotatable cam comprises a heart-shaped cam including two engagable lobes on a first half of said rotatable cam and a pair of converging surfaces on a second half of said rotatable cam.

6. The orthopedic device of claim 5, wherein said rotatable cam is disposed between two thru-bores, each bore containing positionable element coupled to said implant member.

7. The orthopedic device of claim 1, wherein said positionable element comprises a cantilevered arm coupled at a first end to said orthopedic device.

8. The orthopedic device of claim 1, wherein said implant member comprises a cervical plate.

9. The orthopedic device of claim 1, wherein said positionable element comprises a back out projection projecting toward said thru-bore.

10. The orthopedic device of claim 2, wherein said split ring further comprises:
a circular ring body;
a back out projection member projecting from a top of said circular ring body toward said thru-bore; and
an annular retention flange projecting from a bottom of said circular ring body away from said thru-bore.

11. The orthopedic device of claim 10, further comprising:
an annular ring retention undercut defined in said thru-bore;
wherein said annular ring retention undercut flange is configured to receive and retain said annular retention flange when said split ring is in an expanded state.

12. A cervical plate system, comprising:
a cervical plate member defining at least one thru-bore;
a compressible member coupled to said cervical plate member about said thru-bore, wherein said compressible member selectively defines an entry diameter of said thru-bore; and
an actuator coupled to said cervical plate member adjacent to said at least one thru-bore;
wherein said actuator is configured to selectively impart a force on said compressible member to vary said entry diameter.

13. The cervical plate system of claim 12, wherein said compressible member comprises a split ring.

14. The cervical plate system of claim 12, wherein said compressible member comprises a compliant cantilevered member coupled to said cervical plate adjacent to said thru-bore.

15. The cervical plate system of claim 12, wherein said actuator comprises a rotatable heart-shaped cam including two engagable lobes on a first half of said rotatable cam and a pair of converging surfaces on a second half of said rotatable cam.

16. The cervical plate system of claim 15, wherein said rotatable cam is disposed between two thru-bores, each bore containing compressible member coupled to said implant member.

17. The cervical plate system of claim 12, wherein said compressible member comprises a back out projection projecting toward said thru-bore.

18. The cervical plate system of claim 13, wherein said split ring further comprises:
a circular ring body;
a back out projection member projecting from a top of said circular ring body toward said thru-bore; and
an annular retention flange projecting from a bottom of said circular ring body away from said thru-bore; and
wherein said cervical plate further includes an annular ring retention undercut defined in said thru-bore, wherein said annular ring retention undercut flange is configured to receive and retain said annular retention flange when said split ring is in an expanded state.

19. A cervical plate system, comprising:
a cervical plate member defining at least two thru-bores;
a compressible member coupled to said cervical plate member about each of said two thru-bores, wherein said compressible members selectively define an entry diameter of said thru-bores and includes a back out projection projecting toward said thru-bore; and
an actuator coupled to said implant member between said at least two thru-bores, wherein said actuator comprises a rotatable heart-shaped cam including two engagable lobes on a first half of said rotatable cam and a pair of converging surfaces on a second half of said rotatable cam;
wherein said actuator is configured to selectively impart a force on said compressible member to vary said entry diameter.

20. The cervical plate system of claim 19, wherein:
said compressible member comprises a split ring including a circular ring body, a back out projection member projecting from a top of said circular ring body toward said thru-bore, and an annular retention flange projecting from a bottom of said circular ring body away from said thru-bore; and
wherein said cervical plate further includes an annular ring retention undercut defined in said thru-bore, wherein said annular ring retention undercut flange is configured to receive and retain said annular retention flange when said split ring is in an expanded state.

21. The cervical plate system of claim 19, wherein said compressible member comprises a compliant cantilevered member coupled to said cervical plate adjacent to said thru-bore.
